(19) United States
(12) Patent Application Publication
(10) Pub. No.: US 2007/0027217 A1
(43) Pub. Date: Feb. 1, 2007

(54) Composition and Method for Preventing Secondary Burn Ischemia

(76) Inventor: H. Paul Ehrlich, Hershey, PA (US)

Correspondence Address: STAINBROOK & STAINBROOK, LLP, 3558 ROUND BARN BLVD., SUITE 203, SANTA ROSA, CA, 95403, US

(21) Appl. No.: 11/421,038

(22) Filed: May 30, 2006

Related U.S. Application Data
(60) Provisional application No. 60/685,138, filed on May 27, 2005.

(51) Int. Cl. A61K 31/192

(52) U.S. Cl. 514/569

(57) ABSTRACT

A cream composition for the topical application of ibuprofen to prevent secondary burn ischemia, inducing changes in the dynamics of blood flow in the region of application. The cream composition includes approximately 0.5 g of 1% ibuprofen cream. A single application of ibuprofen cream to the surface of a skin burn restores vascular patency within the dermis beneath the burn cream to a burn site thwarts burn induced secondary progressive ischemia and the expansion of dermal necrosis.

### CROSS REFERENCES TO RELATED APPLICATIONS

The present application claims the benefit of U.S. Provisional Patent Application Ser. No. 60/685,138, filed May 27, 2005.

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The present invention relates generally to compositions and methods for the treatment of burns, and more particularly to an ibuprofen cream composition and a method of using the same for the prevention of secondary burn ischemia.

2. Discussion of Related Art Including Information Disclosed Under 37 CFR §§1.97, 1.98

Burn injury induces skin loss in two stages: with immediate induced necrosis as a consequence of the direct dissipation of thermal energy and a delayed necrosis resulting from the loss of blood flow to the dermis surrounding the burn. A burn injury becomes deeper and larger with the loss of this surrounding tissue. The delayed necrosis results from vascular occlusion leading to local ischemia. If blood flow returns to these occluded vessels soon after trauma, the tissue will survive and the expansion of tissue loss will not occur. If blood flow is not restored to the surrounding tissue energy that tissue is incorporated into the burn, adding to the volume of tissue loss and burden of repair. Preventing or reversing vascular occlusion and the reestablishment of blood flow reduces the volume of tissue loss by secondary ischemia.

A burn injury, initiates three zones of vascular response radiating out from the initial site of the burn (Jackson, 1953). The skin and dermis irreversibly destroyed by thermal energy is called the zone of coagulation, characterized by dead cells and heat denatured congealed connective tissues, containing occluded vessels. The region adjacent to the zone of coagulation that initially survives the destruction of the heat energy is the zone of stasis. The zone of stasis is the transition region that may either survive or die. Survival of that tissue is dependent upon the integrity of its blood supply. Soon after burn injury, the blood vessels in the dermis bordering on the burn site undergo vascular dilation, causing blood flow stasis. The retardation of blood flow accommodates the deposition of thrombus within the lumen of vessels, which terminates vascular patency. The loss of vessel patency, the cessation of blood flow to the tissues supplied by these vessels, leads to delayed progressive ischemia and tissue necrosis. If soon after burn trauma blood flow is reestablished in these vessels by the restoration of vessel patency, either by preventing or dissolving intravascular thrombus formation, the affected tissue survives. Peripheral to the zone of stasis is the zone of hyperemia that is characterized by elevated blood flow. The zone of hyperactivity is the staging area for wound repair to proceed. If tissue in the zone of stasis retains blood flow and survives, it becomes incorporated into the zone of hyperemia, decreasing the volume and depth of tissue loss.

The conversion of partial thickness burn injuries into full thickness burn injuries is through delayed progressive ischemia, whereby the disruption of a blood supply to sub-dermal appendages containing populations of epidermal cells produces their termination (Noble et al., 1977). The schematic demonstration of progressive ischemia is presented in FIG._1. A second degree burn will heal, becoming re-epithelialized, through the migration of epidermal cells from numerous viable sub epidermal appendages. The distance traveled by these epidermal cell populations is short due to the high density of sub epidermal appendages, such as sweat glands and hair follicles, in skin. A 3^{rd} degree burn lacks viable sub-epidermal appendages and epithelialization is retarded because re-epithelialization is restricted to the epidermal cell populations located at the edges of the burn site. The retarded closure of a third degree burn prolongs the remodeling phase of repair, where hypertrophic scars develop (Tuan and Nichter, 1998; Ehrlich & Kelley, 1992). The mechanism for secondary burn induced expansion of tissue loss is the cessation of blood flow within the zone of stasis. The impedance of the zone of stasis becoming incorporated into the zone of coagulation requires the reestablishment of blood flow, either by preventing or eliminating intravascular thrombi. If thrombus formation is either prevented or reversed (Ehrlich et al., 1987a), progressive ischemia can be averted and vascular patency established in the zone of stasis (Da Costa et al., 1992; Ehrlich et al., 1983, Choi et al., 1993, Choi et al., 1995).

The rat comb burn model facilitates the examination of the changes in blood flow within the zone of stasis (Ragas and Ehrlich, 1992). A row of four full-thickness burns (“burn sites”) are separated by three rows of unburned skin (“interspaces”). After 24 hours the 4 separate small burns in the untreated comb burn, progress into a single large burn, where the unburned interspaces become incorporated into the burn. The inclusion of the interspaces into the burn represents a 27% increase in surface area of lost tissue. The unburned skin of the interspaces dies because blood flow is terminated. The cessation of blood flow is documented by the absence of yellow latex filled blood vessels in vascular casts. Interventions that salvage vessel patency within the “interspaces” of comb burns are reported. Lazaroids, modified steroids, inhibit the generation of oxygen free radical by chelating iron. The systemic introduction of Lazaroids into comb burned rats prevented vascular occlusion within the interspaces (Choi et al. 1993). Blocking the infiltration of neutrophils into the “interspaces” by the systemic application of a monoclonal antibody directed to CD 11b/CD 18, a leukocyte surface receptor required for cell adhesion and extravasation, preserved blood flow and restored vascular patency (Choi et al., 1995). There appears to be a time window of opportunity for reversing vascular occlusion within the comb burn model. With systemic lazaroids the window is 1 hour post burn (Choi et al. 1993), while the intravenous infusion of an antibody directed to leukocyte surface receptor CDIIb/CD18 has a 30 minute window of opportunity (Choi et al., 1995).

A single injection of ibuprofen also preserves blood flow in the comb burn “interspaces” (Ragas and Ehrlich, 1992). The mechanism for the restoration of vascular patency by ibuprofen is not related to the inhibition of the arachidonic acid cascade (Ehrlich et al., 1983, Ehrlich, 1984b). Rather the mechanism appears to involve the promotion of fibrinolysis and the clearance of intravascular thrombi (Ehrlich, 1984a). Here the local application of ibuprofen in a cream base to a comb burn is investigated and its effect upon yellow latex perfusion of patent blood vessels is reported.

Various topical compositions containing ibuprofen have been proposed for the treatment of cutaneous disorders. Exemplary patents disclosing related art include:

U.S. Pat. No. 5,976,566, to Samour et al., which teaches a stable topical composition effective for the transdermal application of ibuprofen or other non-steroidal anti-inflammatory drug (“NSAID”) compounds by the application of the composition to the skin. The preferred composition is an ibuprofen containing alcoholic or aqueous alcoholic composition which comprises, on a weight basis, of the total composition: a therapeutically effective amount of ibuprofen in the form of its pharmacologically acceptable salt; a skin penetration enhancing effective amount of a C.sub.7 to C.sub.14-hydrocarbyl substituted 1,3-dioxolane, 1,3-dioxane or acetal; 0 to about 18% of glycol having from 3 to 6 carbon atoms; at least 40% of volatile alcohol selected from the group consisting of ethanol, propanol and mixture thereof; 0 to about 40% water; and, base to provide a pH in the range of from about 6 to about 8; and optionally, a gelling agent effective to thicken the composition to avoid or minimize run-off when applied to the skin. Preferably the ingredients are included in the formulation in the following ranges: from about 2 to 10% ibuprofen; from about 2 to 15% of the enhancer wherein the hydrocarbyl group substituent has from about 7 to 10 carbon atoms; from about 0 to 15% propylene glycol; from about 55 to 70% ethanol, isopropanol or mixture thereof; from about 4 to 35% water; and, base to provide a pH in the range of from about 6.5 to about 7.5; and 0 to about 2% of cellulosic thickener.

U.S. Pat. No. 6,562,326, to Miller, discloses a topical compositions and methods of using them for the alleviation of the symptoms associated with burns of human skin. The composition comprises a therapeutically effective amount of a combination of an anesthetic and a surfactant. Varying amounts of anesthetic and surfactant are used to achieve efficacious results, e.g., for anesthetic concentrations of from about 0.05% to 25% by weight, preferably 0.25% to 10% by weight, and most preferably 1% to 5% by weight, and for surfactant, concentrations of from about 0.05% to 50% by weight, 1% to 10% by weight, and 0.5% to 5% by weight

U.S. Pat. No. 4,185,100 to Rovee, et al., describes a pharmaceutical composition for topical treatment of cutaneous disorders or disruptions characterized by skin inflammation or hyperproliferative epidermal activity comprises the combination of a topically active anti-inflammatory corticosteroid and an NSAID agent which is an inhibitor of prostaglandin synthetase selected from the group consisting of the hydratropic acid derivatives; acetylsalicylic acid; the pyrazolone derivatives; the fenamic acid derivatives; the aroyl-substituted pyrroles and the substituted arylacetohydroxamic acids in a pharmaceutically acceptable topical vehicle.

U.S. Pat. No. 6,284,797, to Rhodes, teaches a topical therapeutic composition containing capsaicin, which is an extract of peppers or chiles and which is a potent local pain killer. The ointment also includes a norepinephrine inhibitor and preferably a vasodilator which act to promote blood circulation in the treatment area and thereby promote healing of tissues in the treatment area. The ointment also includes a local pain killer, such as ibuprofen, to offset the irritating effects of the capsaicin and a promoter of transcutaneous absorption. It is suggested that the composition can be employed in the treatment of sunburn.

The importance of mainting vascular integrity to prevent tissue necrosis and reperfusion injury after traumatic injury has been recognized. U.S. Pat. No. 5,489,581 to Daynes, et al, discloses a method for preventing or reducing ischemia following injury, such as reperfusion injury following ischemia, cellular damage associated with ischemic episodes, such as infarctions or traumatic injuries, and thus prevention or reduction of consequent progressive necrosis of tissue associated with such ischemia. The therapeutic effect is achieved by administering DHEA or DHEA derivatives to a patient as soon as possible after the injury.

Finally, U.S. Pat. No. 6,562,326 to Miller, describes a method of treating burns that includes applying a topical composition having as the active ingredients an anesthetic and a surfactant. The anesthetic is preferably tetracaine in a concentration of from about 1% to 2% by weight and the surfactant is preferably sodium lauryl sulfate in a concentration of from about 0.5% to about 5.0% by weight.

The foregoing patents reflect the current state of the art of which the present inventor is aware. Reference to, and discussion of, these patents is intended to aid in discharging Applicant's acknowledged duty of candor in disclosing information that may be relevant to the examination of claims to the present invention. However, it is respectfully submitted that none of the above-indicated patents disclose, teach, suggest, show, or otherwise render obvious, either singly or when considered in combination, the invention described and claimed herein. However, it is respectfully submitted that none of the above-indicated patents disclose, teach, suggest, show, or otherwise render obvious, either singly or when considered in combination, the method of preventing of preventing secondary burn ischemia through the use of topically applied ibuprofen, as described and claimed herein.

### BRIEF SUMMARY OF THE INVENTION

The present invention is a topical cream composition containing ibuprofen as an active ingredient. The composition is employed to reduce and prevent secondary burn ischemia. The cream composition includes approximately 0.5 g of 1% ibuprofen cream. A single application of ibuprofen cream to the surface of a skin burn restores vascular patency within the dermis beneath the burn cream to a burn site thwarts burn induced secondary progressive ischemia and the expansion of dermal necrosis.

It is an therefore an object of the present invention to provide a new and improved composition for preventing secondary burn ischemia that is inexpensive to the user.

It is another object of the present invention to provide a new and improved composition for the prevention of secondary burn ischemia that is topically applied.

A further object or feature of the present invention is a new and improved composition for the prevention of secondary burn ischemia that contains, as active ingredients, only common and readily available drugs.

An even further object of the present invention is to provide a novel composition for preventing secondary burn ischemia that is relatively stable.

Other novel features which are characteristic of the invention, as to organization and method of operation, together with further objects and advantages thereof will be better understood from the following description considered in connection with the accompanying drawings, in which preferred embodiments of the invention are illustrated by way of example. It is to be expressly understood, however, that the drawings are for illustration and description only and are not intended as a definition of the limits of the invention. The various features of novelty that characterize the invention are pointed out with particularity in the claims annexed to and forming part of this disclosure. The invention does not reside in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the devising other compositions and methods for carrying out the several purposes of the present invention. It is important, therefore, that the claims be regarded as including such equivalent constructions insofar as they do not depart from the spirit and scope of the present invention.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawings wherein:

FIG. 1 is a photograph showing the gross appearance of a 24-hour old comb burn on the dorsum of a rat. The burns in the bottom row were placebo treated burns and the burns in the top row were treated with an ibuprofen composition. The stars indicate interspaces that show some discoloration and the arrow head shows an interspace between two burns on the ibuprofen treated rat that showed partial tissue bleeding.

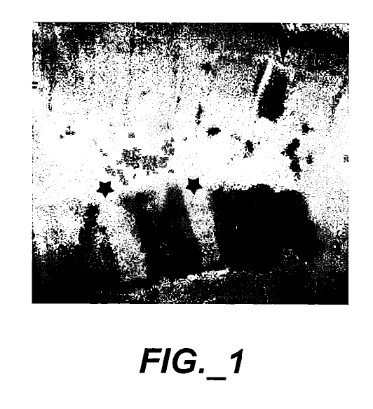

FIG. 2A is a photograph showing the vascular cast of an untreated one day old rat comb burn showing the absence of patent vessels between burn sites.

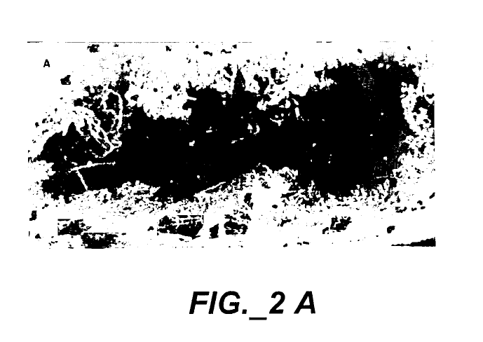

FIG. 2B is a photograph showing the vascular cast of a one day old burn treated with ibuporfen, showing the existence of patent vessels between burn sites.

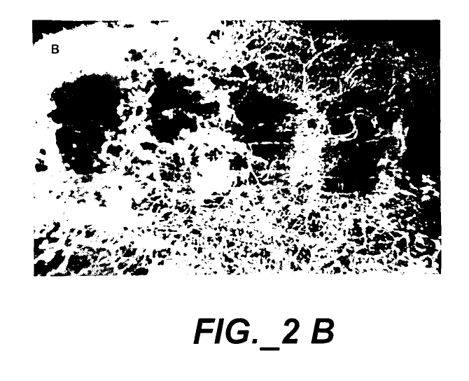

### DETAILED DESCRIPTION OF THE INVENTION

Methods: Male Sprague Dawley rats weighing between 300 and 400 g were anesthestized with halothane, their backs were clipped. The brass comb was made from a rectangular brass bar 55×19×19 mm containing three 5 mm transverse notches along one surface. The brass comb was equilibrated in boiling water, blotted dry then balanced on the rats back parallel to the spine for 20 seconds. No pressure was applied to the brass comb in order to ensure the uniformity of the burns made on each rat. A second identical comb burn was made on the other side of the mid line, after re-equilibrating the brass comb in boiling water. The brass comb produced a parallel row of four rectangular 10×19 mm full thickness burns separated by 5×19 mm “interspaces”. In each rat the two rows of burns represented less then 4 percent of the total body surface area.

The study included 7 rats with comb burns, where the left side received ibuprofen cream and the right side received the cream base alone. To document the amount of cream applied to the comb burn surface, the 1% ibuprofen cream and cream base containers were weighted to the nearest mg before application and re-weighted after application creams to comb burns. The difference in weight was the amount of cream applied to the comb burn. It was found that about 500 mg (5 mg of ibuprofen) of cream was applied to each comb burn. The creams were applied to the surface of each comb burn immediately after burn injury by rubbing 50 times over the surface with an index finger. The comb burns were left undressed and each rat returned to his cage upon waking from anesthesia.

As previously described vascular yellow latex casts were made at 24 hours after burn injury (Ehrlich et al., 1983). Briefly, the rat was placed under halothane anesthesia, his thoracic cavity opened and his heart exposed. A plastic catheter was inserted and secured in the aorta via an incision made in the left ventricle wall of the heart. The right atrium was excised to allow the efflux of fluid from the perfused vasculature. A flask of 10% formalin solution attached to the secured catheter was located at a fixed height of 100 cm. The 10% formalin was passed through vasculature via the catheter until the liquid eluting from the right atrium was void of red blood cells and ran clear. Yellow latex, Microfil® compound, (Flow Tech, Inc., Carver, Mass.) was mixed following manufacturer's instructions and about 20 ml was perfused through the secured catheter with a 25 ml syringe. When the effluent from the right atrium became yellow, the patent vessels were filled and perfusion was terminated. The rat was placed in a refrigerator overnight to allow the latex to cure. The next morning the dorsum skin that included the pair of comb burns was excised and placed in ascending concentrations of ethyl alcohol (50 to 100%) to dehydrate the skin. Over a five (5) day period the skin became completely dehydrated and was submerged in methyl salicylate, which rendered the skin transparent while the latex filled vessels were bright yellow. While submerged in the methyl salicylate, the vascular casts were photographed with digital camera.

Results: A pair of comb burns on each side of the dorsum of rat at 1 days were photographed (see FIG._2A). There was little difference in the gross appearance between the row of “interspaces” and the “burn sites” in the ibuprofen cream treated comb burn on the “I”, row, compared to the placebo cream treated comb burn or “P” row. There was a slight redness in the interspaces in some of the ibuprofen treated comb burns but it was not a consistent observation.

Vascular casts of comb burns revealed no yellow filled blood vessels within the burn injury sites. All the vessels were completely occluded in both ibuprofen treated and untreated comb burns (FIG._2B). However, the vascular casts of the interspaces from the ibuprofen treated comb burn showed yellow latex within vessels. The placebo treated comb burn interspaces had the same appearance as the burn sites, where yellow latex was absent from the vessels. The direct destruction of dermis by thermal injury resulted in total occlusion of all the blood vessels in that region. When examined 24 hours after burn injury blood vessels within the interspaces also became occluded in the placebo treated comb burns. The topical application of ibuprofen cream prevented occlusion of a major portion of the blood vessels within “interspaces”. The application of 500 mg of 1% ibuprofen cream (total of 5 mg of ibuprofen) on to a burn area of 1.9 cm by 5.5 cm (10.4 cm² area), representing a dose of about 2 mg of ibuprofen per cm² of burn wound surface, prevented occlusion of blood vessels within the interspaces.

Discussion: The rat comb burn model facilitates the study of changes in the patency of blood vessels within the burn and the dermis beneath the burn. Burn induced delayed ischemia generated tissue necrosis occurs beneath a burn in the zone of stasis. In the comb burn model a portion of the zone of stasis is located on the surface of the skin, which are the interspaces between the square burn wounds. In the comb burn the occlusion of vessels in the 3 interspaces instigates their incorporation into the 4 burn wounds generating a single larger burn. If the prevention of blood vessel occlusion or the reestablishment of blood flow in the interspaces occurs, then the skin in the interspaces survives and remains viable. Yellow latex filled patent vessels are expected in viable dermis. Vascular casts from ibuprofen treated comb burns have yellow latex patent vessels localized in their interspaces. Such vascular casts indicate viable skin between the burns and the restoration of vascular perfusion. The interspaces of ibuprofen treated comb burns will not undergo necrosis and the volume of burn destroyed dermis does not increase through delayed ischemia.

The identity of patent vessels in the interspaces of comb burns is reported to correlate with blood flow measurements (Ragas and Ehrlich, 1992). The preservation of blood flow in the zone of stasis preserves the viability of cell populations within sub-epidermal appendages. Epidermal cells that survive within sub-epidermal appendages are responsible for the rapid closure of second degree burns. The pattern of numerous sub-epidermal appendages found in skin make many loci for the initiation of re-epithelialization and the rapid resurfacing of the lost epidermis destroyed by burn trauma. If blood flow is not restored or maintained within the zone of stasis, sub-epidermal appendages do not survive and a second-degree burn injury progresses into a third-degree burn injury. In third degree burns the lack of epidermal cells from the sub-epidermal appendages participating in re-epithelialization retards wound closure. The longer a burn wound remains open, the greater the chances for hypertrophic scarring. Promoting vascular integrity within the zone of stasis of a second degree burn wound, promotes rapid closure, which reduces the chances of infection and excess scarring.

The intravenous infusion of ibuprofen into a burned hamster model shows a reduction in the vasodilation response to burn injury (Da Costa et al., 1992). Preventing vasodilatation maintained the microcirculation and eliminates edema in that animal model. In the rat comb burn model, the injection of ibuprofen restores vessel patency and blood flow in the “interspaces” (Ragas and Ehrlich, 1992). The restoration of vasculature patency and blood flow in ibuprofen treated burn wounds is not by the prevention of intravascular formation of thrombi, rather it is by the breaking up of the thrombi (Ehrlich et al. 1987b). Rat comb burns injected with ibuprofen immediately after burn trauma demonstrated the restoration of blood flow within “interspaces” starting at 16 hours (Ragas and Ehrlich, 1992). The delay of 16 hours in the return of blood flow does not support the notion that ibuprofen prevents the deposition of fibrin. If ibuprofen prevented thrombi from forming, then the recovery of vascular patency would occur be soon after treatment. The 16 hour delay in restoring vascular patency implies that the disruption of formed thrombi by fibrinolysis is involved. There is little evidence that ibuprofen prevention of the arachidonic acid cascade plays a role in the restoration vessel patency in burn injuries (DelBeccaro., 1980; Ehrlich, 1984b). As an example indomethacin like ibuprofen, is a potent inhibitor of cyclooxygenases and blocks the arachidonic cascade, but does not prevent vascular occlusion and loss of blood flow in burns (Ehrlich, 1984a).

There is a case report of a severely burned patient benefiting from systemic ibuprofen. A burned victim, who was taking ibuprofen for his arthritis condition, received a 38% full thickness burn. He failed to show the expected hypermetabolic response during his recovery (Waymack et al., 1990). The speculation is that systemic ibuprofen attenuates of the hypermetabolic response to thermal injury by reducing the severity of body temperature elevation in these patients (Wallace et al. 1992). Another interpretation is ibuprofen prevention of tissue loss by diminishing progressive ischemia and delayed necrosis reduces the ibuprofen treated burn patient's necrotic burden.

The topical application of ibuprofen or a derivative (unreported finding with flurbiprofen cream) to a thermal injury will prevent the deepening of that injury and the conversion of partial thickness superficial burn wounds into a more severe full thickness burn wounds. The safety of topically applied ibuprofen is bolstered by it availability as an over-the-counter pill. In some people a side effect of orally administered ibuprofen is an upset stomach. The application of ibuprofen as a topical treatment for second-degree burns would eliminate the stomach upset side effect in some individuals. Limiting the depth and size of a second-degree burns is expected to reduce the time of wound closure, improve aesthetic results and eliminate the need for skin grafting.

It should be noted that those skilled in the art will appreciate that the conception upon which this disclosure is based readily may be utilized as a basis for preparing other compositions, methods and systems for carrying out the several purposes of the present invention. It is important, therefore, that the instant specification be regarded as including such equivalent compositions insofar as they do not depart from the spirit and scope of the present invention.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. A method of preventing secondary burn ischemia, comprising the steps of applying an ibuprofen cream composition to the affected wound area.

2. The method of claim 1, wherein the ibuprofen cream composition is applied in a concentration of about 2 mg of ibuprofen per cm² of burn wound surface.

3. A method of preventing secondary burn ischemia, comprising the steps of:
providing approximately 500 mg of 1% ibuprofen cream (5 mg of ibuprofen);
applying the composition to a burn area in an amount representing a dose of about 2 mg of ibuprofen per cm² of burn wound surface.

4. A composition for the prevention of secondary burn ischemia, comprising a therapeutically effective amount of ibuprofen, in the range of about 0.1 to 50 mg in a pharmaceutically acceptable carrier.

5. The composition of claim 4, where said pharmaceutically acceptable carrier is a cream base.
